(19) United States
(12) Patent Application Publication
(10) Pub. No.: US 2012/0123193 A1
(43) Pub. Date: May 17, 2012

(54) METHOD OF ASSESSING EMBRYO OUTCOME

(75) Inventors: James T. Posillico, Chester, NJ (US); Dionisios Sakkas, Hamden, CT (US); Mark Jeffrey Henson, Quaker Hill, CT (US); Lucy Botros, Hamder, CT (US)

(73) Assignee: BWT BIOMETRICS, LLC, NEWARK, DE (US)

(21) Appl. No.: 13/384,240

(22) PCT Filed: Jul. 15, 2010
(86) PCT No.: PCT/US10/42104

§ 371 Date: Jan. 31, 2012

Related U.S. Application Data
(60) Provisional application No. 61/226,215, filed on Jul. 16, 2009.

(51) Int. Cl. A61B 17/435; G01N 30/00; G01N 37/00; H01J 49/00

(52) U.S. Cl. 600/34; 250/282; 73/61.52; 73/61.43

(57) ABSTRACT

Non-invasive methods of predicting embryo outcome by analyzing disclosed markers in embryo culture media.

### RELATED APPLICATION

The present application claims the benefit of and priority to U.S. Provisional Application No. 61/226,215, filed on Jul. 16, 2009, the entire contents of which are incorporated by reference herein.

### BACKGROUND

In vitro fertilization offers hope for conception for couples who are subfertile, but is limited by low success rates. Typically only a fraction of the embryos generated by in vitro fertilization develop to full term; consequently multiple embryos are transferred into a single recipient to increase the likelihood of a resulting pregnancy. However, transfer of multiple embryos often leads to multiple pregnancies, which results in increased risk of medical complications for mothers as well as infants. To limit these medical complications, a particularly attractive alternative is to transfer a single embryo.

### SUMMARY

The present invention encompasses the recognition that tools to help predict embryo outcome would allow transfer of a single embryo and consequently reduce the risk of multiple pregnancies. Disclosed are novel markers that can be used to predict embryo outcome. Markers disclosed herein can be assessed noninvasively, for example, by analyzing any culture media in which embryos are grown before transfer.

### BRIEF DESCRIPTION OF THE DRAWING

FIG. 1 depicts the statistical comparisons used in Example 1.

FIG. 2 shows box plots of potential markers identified by a two-sample t-test.

FIG. 3 shows box plots of potential markers identified by a matched pair t-test.

### DEFINITIONS

As used herein, the terms “biomarker” and “marker” are used interchangeably and refer to their meaning as understood in the art. The term can refer to an indicator that provides information about, among other things, a process, condition, developmental stage, or outcome of interest (e.g., an embryo's viability and/or likelihood of having a positive outcome (e.g., of implanting into a uterine wall, of developing to a certain stage, of fully developing through birth, of fully developing through birth as an infant with no chromosomal abnormalities, etc.) after being transferred to a uterine tract of an appropriate host). In general, the value (e.g., amount) of such an indicator is correlated with a process, condition, developmental stage, or outcome of interest. The term “biomarker” or “marker” can also refer to a molecule that is the subject of an assay or measurement the result of which provides information about a process, condition, developmental stage, or outcome of interest. For example, an altered level of a particular compound (e.g., a metabolite or derivative thereof and/or small molecule) in culture media can be an indicator that an embryo is likely to have a certain outcome with respect to viability and/or development. An altered level of the compound and the compound itself can all be referred to as “biomarkers” or “markers.”

As used herein, the term “egg” or “ovum” (plural “ova”) refers to a female gamete that, in normal biology, can be fertilized by a spermatocyte to give rise to an organism. The term encompasses fully functional as well as developmental abnormal eggs or ova.

As used herein, the term “embryo” refers to an organism in the early stages of growth and differentiation. In mammals including humans, the term “embryo” encompasses an organism from as early a stage as fertilized oocyte/ovum (also referred to as “zygote”) to, in humans, the beginning of the third month of pregnancy.

As used herein, ther term “gamete” refers to a cell involved in reproduction, e.g., a sex cell.

As used herein, the term “oogonia” refer to stem cells that can develop into oocytes and eventually into ova.

As used herein, the term “oocyte” (also know as “ovocyte” or “ocyte”) is a female germ cell that gives rise to an ovum (egg). The term “oocyte” as used herein encompasses immature oocytes at all developmental stages after precursor oogonia cell up to an ovum that can be fertilized, including both primary oocytes (which have undergone a first meiotic division) and secondary oocytes (which have undergone a second meiotic division).

As used herein, the term “spermatozoa” (also known as “sperm”) refers to male germ cells that, in normal biology, can fertilize an egg to give rise to an organism. The term encompasses fully functional as well as developmentally abnormal spermatozoa. For example, spermatozoa that cannot fertilize an egg without artificial assistance (e.g., through use of a technique such as intra-cytoplasmic sperm injection) are included in the term “spermatozoa”.

### DETAILED DESCRIPTION OF SOME EMBODIMENTS

Methods disclosed herein generally comprise steps of: providing a sample of culture media in which an embryo has been cultured in vitro; measuring in the sample of culture media, amount of one or more markers for embryo outcome; and characterizing, on the basis of amount of the one or more markers, whether the embryo is likely to have a positive outcome. In certain embodiments, the one or more markers comprise a compound selected from the group consisting of 4-methyl-2-oxopentanoate, glycylglutamate, p-cresol sulfate, phenylalanine, tryptophan, valine, and combinations thereof.

### I. Embryos

Embryos that can be assessed using methods of the invention can be generated by any of a variety of methods known in the art.

In some embodiments, embryos develop from zygotes generated by in vitro fertilization (IVF). In IVF, oocytes or ova are typically inseminated in vitro by placing them in a suspension of spermatozoa. Fertilized oocytes or ova are then cultured in vitro to develop as embryos, which can then be transferred into the uterine tract of a host female.

In some embodiments, embryos develop from zygotes generated by intracytoplasmic sperm injection (ICSI) of an oocyte or ovum. ICSI is an increasingly popular method of assisted reproductive technology (ART), as it allows an oocyte or ovum to be fertilized independently of the motility or morphology of the single spermatozoa injected. Mature spermatozoa as well as immature spermatozoa (e.g., those retrieved surgically from the epididymis and testis) can be used in ICSI. ICSI offers an ability to generate an embryo using sperm that would normally not be able to fertilize an oocyte or ovum encapsulated in its zona pellucida and surrounded by accompanying cumulus cells. In ICSI, a single sperm is injected mechanically into an oocyte or ovum using a small-bore pipette or microinjection needle.

In some embodiments, embryos develop from zygotes generated by nuclear transfer (NT) to an enucleated oocyte or ovum. Nuclear transfer is often used in the creation of transgenic animals, for example, farm animals used as livestock. Cells whose nuclei can be used in nuclear transfer procedures include stem cells (e.g., embryonic stem cells and tissue stem cells), progenitor cells, and somatic cells (e.g., differentiated cells of a particular tissue type).

Oocytes and ova can be obtained from any female animal that produces them and from whom an embryo of the same species is desired. Typically, oocytes and/or ova are surgically removed from donor females. In some embodiments, oocytes and/or ova are obtained from mammalian females. In some embodiments, oocytes and/or ova are obtained from human females.

In some embodiments, the female from which the oocyte and/or ova is obtained is injected with hormones in order to stimulate oocyte maturation and/or release of oocyte(s) from follicles; such hormone stimulation often results in the maturation and/or release of more oocytes than would normally be matured and/or released in a natural ovulatory cycle and is sometimes known as “superovulation.” Hormones for stimulating oocyte maturation are known in the art, commercially available, and include, but are not limited to, human chorionic gonadotropin and luteinizing hormone. Hormones for stimulating release of oocytes from follicles are known in the art, commercially available, and include, but are not limited to, follicle stimulating hormone and pregnant mare serum gonadotropin.

In some embodiments, immature oocytes are obtained from the female and the oocytes are matured in vitro.

In some embodiments, oocytes and/or ova to be used to generate embryos (e.g., to be fertilized, injected, or used in nuclear transfer procedures) are subjected to one or more procedures that facilitate ease of using such oocytes and/or ova in certain procedures. For example, oocytes and/or ova may be freed of associated cumulus cells. Removal of cumulus cells can be accomplished, for example, using enzymes such as hyaluronidase. Alternatively or additionally, oocytes and/or ova can be removed from their zona pellucida. Removal of zona pellucida can be accomplished, for example, mechanically (e.g., using microdissection), enzymatically (e.g., using trypsin or pronase, a commercially available mixture of proteinases), and/or using an acidic solution. Alternatively or additionally, one or more holes can be created in the zona pellucida of oocytes and/or ova to facilitate, for example, injection of the oocyte and/or ovum. Creation of a hole can be accomplished, for example, using an acid solution applied through a fine micropipette (e.g., a glass micropipette) and/or mechanically splitting the zona using tools under control of a micromanipulator with or without prior softening with brief exposure to enzymes (e.g., trypsin or pronase).

Oocytes and/or ova are activated upon fertilization. However, in some procedures involving artificial insemination (e.g., ICSI), certain steps are bypassed that would normally activate the oocyte or ovum. In some embodiments, oocytes and/or ova are activated artificially. Activation of oocytes and/or ova can be accomplished artificially using, for example, energetic suction of the ooplasm prior to sperm nucleus insertion and/or exposure to chemicals (e.g., calcimycin also known as A23187).

Spermatozoa (used, for example, in in vitro fertilization and in intracytoplasmic sperm injection procedures) can be obtained from any male animal that produces them and from whom an embryo of the same species is desired. Spermatozoa may be obtained by, for example, ejaculation and/or surgical removal from the donor male. In some embodiments, spermatozoa are obtained from mammalian males. In some embodiments, spermatozoa are obtained from human males.

Spermatozoa may undergo certain procedures after they are obtained from donors. In some embodiments, spermatozoa are activated (also known as “capacitated”) in vitro using, for example, calcium ionophores and/or exposure to cumulus cells and/or to progesterone (which is released from cumulus cells).

### II. Embryo Culture

Once zygotes are generated, they are generally cultured in vitro in appropriate growth media to develop into embryos. Growth media generally comprise essential amino acids (i.e., phenylalanine, valine, threonine, tryptophan, isoleucine, methionine, leucine, and lysine for human adults; cysteine (or sulphur-containing amino acids), tyrosine (or aromatic amino acids), histidine, and arginine may also be considered essential for infants and growing children). In some embodiments, growth media further comprise non-essential amino acids. In addition to amino acids, typical components of growth media include, but are not limited to, metabolic precursors and other nutrients (e.g., glucose, sodium pyruvate, alanyl-glutamine, lactate, glutathione, etc.), derivatives of amino acids (e.g., taurine, alanyl-glutamine) and salts (e.g., sodium chloride, potassium chloride, magnesium sulfate, calcium lactate, sodium bicarbonate, sodium citrate, and combinations thereof). Other components typically used in growth media for growing embryos include vitamins (e.g., choline chloride, folic acid, i-Inositol, nicotinamide, pyridoxine, riboflavin, thiaminetaurine, etc.), antibiotics (e.g., gentamicin) that prevent or reduce growth of microorganism, and additives (e.g., phenol red).

A variety of suitable growth media are commercially available for in vitro culture of embryos, including embryos intended to be later transferred into recipient females to develop further. For example, a number of commercial media are available (including Sage Cleavage and Blastocyst Media, Vitrolife G1.5 and G2.5, Cook Sydney IVF Media, Medicult ISM1&2, Global IVF media) and are used for culture of embryos in vitro.

Embryos are generally cultured in appropriate culture dishes and/or plates (e.g., petri dishes, plates with wells, etc.), which can be made from a variety of materials such as glass and/or polystyrene. Typically, the volume of media in which embryos are cultured is below a certain range. In some embodiments, the volume is less than 1000 μL, 950 μL, 900 μL, 850 μL, 800 μL, 750 μL, 700 μL, 650 μL, 600 μL, 550 μL, 500 μL, or less. In some embodiments, the volume is less than 500 μL, 480 μL, 460 μL, 440 μL, 420 μL, 400 μL, 380 μL, 360 μL, 340 μL, 320 μL, 300 μL, 290 μL, 280 μL, 270 μL, 260 μL, 250 μL, 240 μL, 230 μL, 220 μL, 210 μL, 200 μL, 190 μL, 180 μL, 170 μL, 150 μL, 140 μL, 130 μL, 120 μL, 110 μL, 100 μL or less. In some embodiments, the volume is less than 100 μL, 95 μL, 90 μL, 85 μL, 80 μL, 75 μL, 70 μL, 65 μL, 60 μL, 55 μL, 50 μL, 45 μL, 40 μL, 35 μL, 30 μL or less. In some embodiments, the volume is less than 30 μL, 29 μL, 28 μL, 27 μL, 26 μL, 25 μL, 24 μL, 23 μL, 22 μL, 21 μL, 20 μL, 19 μL, 18 μL, 17 μL, 16 μL, 15 μL, 14 μL, 13 μL, 12 μL, 11 μL, 10 μL or less.

In some embodiments, for example in embodiments where the volume is less than about 250 μL, embryos are cultured in very small wells (e.g. in wells of 96-well plate) and/or in microdrops of media in culture dishes or plates. Microdrops can be deposited onto a surface. In some embodiments, oil (e.g., mineral oil) is layered over the surface containing microdrops, which may reduce evaporation of liquid from the microdrop in the incubator.

In some embodiments, a single embryo is cultured in each dish, well, or microdrop. In some embodiments, an embryo to be analyzed is cultured alongside an empty (e.g., without an embryo) dish, well, or microdrop of media; the media cultured without an embryo can be used as a control.

Typically, embryos are cultured in an incubator that allows control of temperature and/or gas levels (e.g., of CO₂ and/or O₂). Incubators that may be used in accordance with the invention include, but are not limited to, traditional laboratory incubators, bench top incubators (such as, for example MINC incubators (Cook, Planar), and microfluidic chamber setups. Ideal temperature ranges and CO₂/O₂ concentrations for growing a given type of embryo are known in the art. For example, embryos are typically cultured in a temperature range between about 34.0° C. and about 39.5° C. Ideal temperatures for culturing embryos may depend on the species. In some embodiments, embryos are cultured at a temperature of about 37° C. Typical concentrations of CO₂ suitable for culturing embryos range from about 5% to about 6%. In some embodiments, embryos are cultured at a CO₂ concentration of about 5%. Typical concentrations of O₂ suitable for culturing embryos range from about 5% to about 20% (air). In some embodiments, O₂ concentration is lowered in incubators by increasing N₂ concentrations. In some embodiments, embryos are cultured at an O₂ concentration of about 5%.

In some embodiments, embryos are cultured and/or handled in sterile or near-sterile conditions. For example, certain procedures (e.g., removal of growth media, addition of new growth media, preparation for embryo transfer) may be done in a flow hood and/or in a sterile laboratory room.

### III. Analyses

### A. Preparation of Samples

Samples of media in which embryos have been cultured can be obtained and prepared for analysis.

In some embodiments, samples are subjected to one or more procedures such as concentration, removal of impurities, dilution, extraction of compounds, etc. Such procedures may facilitate handling of samples and/or subsequent analyses.

In some embodiments, samples are stored for a period of time before analysis. In some embodiments, samples are stored without being processed in any manner. In some embodiments, samples are stored after being subject to one or more procedures such as extraction of compounds, small molecules, and/or metabolites. In some embodiments, samples are frozen (e.g., at less than −10° C., less than −20° C., less than −50° C., less than −70° C. than −80° C., less than −90° C., less than −100° C., less than −110° C., less than −120°C., less than −130° C., less than −140° C., less than −150° C., less than −160° C., less than −170° C., less than −180° C., less than −190° C., less than −200° C., less than −210° C., less than −220° C., less −230° C., less than −240° C., or less) during at least some of the period of time in storage. In some such embodiments, samples are stored in liquid nitrogen.

B. Measuring Amount and/or Detecting Presence of Markers

The present disclosure describes, among other things, a particular application of the principle that amounts and/or presence of certain markers in embryo culture media can be used to predict the likelihood of a positive embryo outcome. A variety of methods of detecting presence of and/or measuring amounts of compounds are known in the art.

In some embodiments, a marker may be identified using chromatography (e.g., gas chromatography (GC) or liquid chromatography (LC)). Exemplary liquid chromatographic techniques include High Performance Liquid Chromatography (“HPLC”), anion exchange chromatography, cation exchange chromatography, ion pair reversed-phase chromatography, single dimensional electrophoresis, multi-dimensional electrophoresis, size exclusion chromatography, affinity chromatography, reverse phase chromatography, capillary electrophoresis chromatography, ion mobility separation, etc.

In some embodiments, a marker may be identified using mass spectroscopy. Without limitation, suitable mass spectroscopic techniques may be based on MALDI (matrix-assisted laser desorption ionization) or ESI (electrospray ionization) or any other ionization method, as well as any suitable detection method, such as ion trap, time-of-flight, or quadrupole analyzers. Exemplary methods are disclosed in the Examples.

In some embodiments, compounds present in media samples are separated before, as, or after they are analyzed. For example, compounds may be separated by chromatography and then identified by mass spectroscopy (e.g., GC-MS or LC-MS, including tandem MS techniques). It is to be understood that these and any other methods described herein do not necessarily require the identity of the marker to be confirmed or even known (e.g., in some embodiments, a marker may be identified based on the presence of characteristic peaks in a chromatograph and/or mass spectrum without determining the identity of the marker).

In some embodiments, a marker may be identified using electrochemical analysis, nuclear magnetic resonance spectroscopy (NMR), fluorescence spectroscopy, refractive index spectroscopy (RI), ultraviolet spectroscopy (UV), infrared spectroscopy (IR) (e.g., near-infrared spectroscopy or Fourier transform infrared spectroscopy), Raman spectroscopy, radiochemical analysis, an immunoassay, Light Scattering analysis (LS), etc. and any combination thereof (including combinations with a chromatographic technique and/or a mass spectroscopic technique). In some embodiments, a marker may be identified using two or more different analytical techniques. In some embodiments, a marker may be identified using two or more sequential analytical techniques. In some embodiments, a marker may be identified using two or more analytical techniques in parallel.

In some embodiments, presence of and/or amount of a marker is assessed in comparison to a control or threshold value, as described further below.

In certain embodiments, data representing amounts of markers in samples are analyzed statistically to determine whether two values are the same or different. A variety of statistical tests and measures of statistical significance are established in the art and may be used in accordance with the invention. Non-limiting examples of commonly used statistical tests for analyzing data that are evenly distributed and/or assumed to be evenly distributed (e.g., parametric tests) include the Student t-test (including one-sample t-tests, two-sample t-tests and matched pair t-tests) and analysis of variance (ANOVA; one-way and two-way or repeated-measures). Non-limiting examples of commonly used statistical tests for analyzing data that are not evenly distributed include the Wilcoxon Rank-Sum test and the Mann Whitney U test. Stringency (e.g., through cutoff values for p-values and/or q-values, as explained below) may be set according to a standard and/or may be set empirically for a given data set. The choice of a statistical test to use may depend on one or more factors including, but not limited to, distribution of the data, type of comparison being performed (e.g., experimental data to a reference value versus two sets of experimental data to each other) and relationship between samples (e.g., matched pairs (such as an experimental sample with a matched control) versus no relationship).

Two indicators of statistical significance are typically used to evaluate data. P-values indicate the probability of obtaining the values that were observed if the null hypothesis were not true. For example, when comparing samples from negative outcome embryos and samples from positive outcome embryos, the null hypothesis can be that amounts of compounds would not differ significantly between the two samples. Lower p-values indicate statistical significance; i.e., increased likelihood that the null hypothesis is not true and should be rejected. q-value indicates the false discovery rate, i.e. a measure of the proportion of false positives that occur when a particular test is considered significant. As with p-values, lower q-values indicate greater significance. In some embodiments, a p-value cutoff is used. In some embodiments, a q-value cutoff is used. In some embodiments, both a p-value and a q-value cutoff are used. In some embodiments, a p-value cutoff of p<0.05 is used. In some embodiments, a more stringent p-value cutoff, e.g., p<0.01, p<0.005, p<0.001, etc. is used. In some embodiments, a q-value of q<0.2 is used. In some embodiments, a more stringent q-value cutoff e.g., q<0.1, p<0.05, p<0.01, etc. is used. Any combination of p-value and q-value cutoff may be used in embodiments where both cutoffs are used, e.g., p<0.05 combined with q<0.2.

### C. Markers

Markers disclosed by the present invention include a number of compounds whose amounts in media samples from cultured embryos can be used as an indication of likelihood of positive outcome. Disclosed markers include a number of markers not present in the original composition of the embryo culture media and/or are modifications or derivatives of essential or non-essential amino acids present in the culture media, including 4-methyl-2-oxopentanoate, glycylglutamate, phenylalanine, p-cresol sulfate, tryptophan, and valine. Alanine and pyruvate can also be asssessed in addition to any of the aforementioned markers.

In some embodiments, presence of the marker in an absolute and/or relative amount greater than that of a control or threshold is used as an indication of likely positive outcome. Disclosed markers whose presence in an amount greater than that of a control or threshold can be used as an indication of likely positive outcome include 4-methyl-2-oxopentanoate, glycylglutamate, phenylalanine, p-cresol sulfate, tryptophan, and valine. In some embodiments, presence of the marker in an amount at least 1.1-fold, 1.2-fold, 1.3-fold, 1.4-fold, 1.5-fold, 1.6-fold, 1.7-fold, 1.8-fold, 1.9-fold, 2.0-fold, or greater as compared to that of a control or threshold is indicative of likely positive outcome.

In some embodiments, presence of the marker in an absolute and/or relative amount less than that of a control or threshold is used as an indication of positive outcome. In some embodiments, presence of the marker in an amount at most 0.9-fold, 0.8-fold, 0.7-fold, 0.6-fold, 0.5-fold, 0.4-fold, 0.3-fold, or less as compared to that of a control or threshold is indicative of likely positive outcome.

In some embodiments, amount of a given marker is compared to that of a control or threshold value. Control values may, for example, be obtained from archived data, from control samples and/or from theoretical calculations of expected values for embryos not likely to have a positive outcome. Control samples from which control values may be obtained include media only controls (e.g., samples from media drops and/or wells without embryos incubated in parallel with embryos to be assessed) and media in which embryos having or likely to have certain outcomes (e.g., positive or negative outcomes) are cultured. Threshold values may be set according to, for example, archived data (e.g., from previous experiments and/or data reported by others) and/or calculated expected values above or below which a likely positive outcome for the embryo is predicted.

### D. Additional Indicators

In some embodiments, the determination of whether the embryo is likely to have positive outcome is made solely on the basis of amount of one or more markers as disclosed herein.

In some embodiments, one or more additional indicators is/are used in combination with markers of the present disclosure to determine whether the embryo is likely to have a positive outcome. Non-limiting examples of other indicators include morphology during zygote and/or early cleavage stages, morphology of pronuclei in fertilized zygotes, cleavage timing, morphology during cleavage stages, and morphology during the blastocyst stage. In some embodiments, one or more other indicators is/are used to select embryos to undergo further culturing and/or subsequent selection using markers of the present disclosure. In some embodiments, one or more indications is/are used at or around the same time to select embryos to undergo further culturing and/or transfer to a uterine tract. In some embodiments, one or more indicators is/are used subsequent to using markers of the present disclosure to select embryos to undergo further culturing and/or transfer to a uterine tract.

For example, zygotes can be examined for a number of features including pronuclei. Features of pronuclei that may be associated with positive outcome include small difference of number of nucleolar precursor bodies (NPBs) in both pronuclei (e.g., differing by three or less NPBs), and coordination in polarization state between pronuclei (e.g., polarized in both pronuclei or not polarized in both pronuclei, but not polarized in one but not the other). Features of pronuclei that are known to be associated with euploidy, and may therefore also be associated with positive outcome, include juxtaposed pronuclei, large-size nucleoli, and polar bodies with small angles subtended by pronuclei and polar bodies. Other features that can be examined in the zygote include presence of a cytoplasmic halo.

Cleavage timing may also be used as an indicator of likelihood of positive outcome. Early cleavage to the two-cell stage (e.g., at 24-27 hours after fertilization) may be used as a favorable indicator. Timing of alignment of pronuclei and nucleoli, appearance of cytoplasm, nuclear membrane breakdown, and/or cleavage to the two cell stage may be used to assess cleavage timing. For example, embryos at a given day after fertilization (e.g., on day 1) may be scored for presence of the above features related to timing, and embryos of a certain score may be deemed early cleavers and to be likely to have a positive outcome.

Morphological characteristics of cleavage stage embryos that may be used as indicators of positive outcome include presence of four or five blastomeres on day 2 and at least seven blastomeres on day 3 after fertilization, absence of multinucleated blastomeres, and less than 20% of fragments on day 2 and day 3 after fertilization.

Morphological characteristics of blastocyst stage embryos that may be used as indicators of outcome include expansion state of blastocoelic cavity and number and cohesiveness of inner cell mass and trophectodermal cells.

In some embodiments, other metabolic indicators may be used in conjunction with disclosed markers. For example, levels of alanine and pyruvate are altered in embryos depending on outcome (see Tables 1 and 2) and either or both may be used as an indicator.

### III. Embryo Transfer

In certain embodiments, methods further comprise transferring the embryo whose likelihood of positive outcome has been assessed to the uterine tract of a recipient.

Embryos may be transferred at different developmental stages or times in culture. Typically, embryos are transferred after 1 day, 2 days, 3 days, 4 days, 5 days, or 6 days in culture. In some embodiments, embryos are transferred at the one-cell stage, the two-cell stage, the four-cell stage, the 8-cell stage, the 16-cell (morula) stage, at the blastocyst stage, or any intermediate stage (e.g., a three-cell embryo that has not completed division to the four-cell stage, a five-cell embryo, a seven-cell embryo, etc.). In some embodiments, embryos are transferred along with their zona pellucida. In some embodiments, embryos are transferred with zona pellucida that are slightly thinned and/or pierced. In some embodiments, embryos are transferred without zona pellucida. For example, in some embodiments, zona pellucida are removed from the oocyte, ovum, or developing embryo during a procedure. In some embodiments, embryos have hatched out of their zona pellucida by the time they are transferred. For example, some embryos may be transferred as blastocyts that have already hatched.

In some embodiments, the recipient is a mammalian female.

In some embodiments in which the female recipient is non-human (e.g., in mice) recipient females are mated with vasectomized males according to a certain schedule prior to an anticipated embryo transfer; such matings may encourage appropriate release of hormones in the female recipient to encourage uterine receptivity and implantation.

The number of embryos transferred to a given recipient female during a given procedure or treatment cycle may be restricted intentionally, e.g. to avoid the likelihood of multiple births. In some embodiments, at most three embryos are transferred during a given transfer procedure or treatment cycle. That is, each embryo is transferred with no more than two other embryos at the same time. In some embodiments, at most two embryos are transferred during a given transfer procedure or treatment cycle. That is, each embryo is transferred with no more than one other embryo at the same time. In some embodiments, a single embryo is transferred during a given transfer procedure or treatment cycle. That is, each embryo transferred is the only embryo transferred into a particular recipient at a given time.

In some embodiments, prior to transfer, embryos deemed to have a high likelihood of a positive outcome are stored for a time period before transfer to the uterine tract of a recipient. Storage can comprise freezing (which in turn can comprising freezing in cryoprotective materials)

### IV. Positive Outcome

Disclosed markers can serve as indicators of positive outcome for embryos being assessed. A positive outcome can indicate embryo viability, likelihood of implantation, active metabolism, more efficient nutrient utilization, etc. In some embodiments, a positive outcome comprises successful implantation into the uterine wall of a female mammal into which the embryo is transferred.

In some embodiments, a positive outcome comprises clinical pregnancy of the female mammal into which the embryo is transferred. Clinical pregnancy can be assessed by any of a variety of techniques well known in the art. For example, urine and/or blood levels of human chorionic gonadotropin (hCG) may be used to indicate clinical pregnancy. hCG levels tend to rise within a few weeks of implantation. Generally, hCG levels above a certain threshold (e.g., at least 25 mIU/mL (milli-international units per milliter)) at a certain time point after fertilization and/or embryo transfer are indicative of clinical pregnancy, and detection of a certain level of hCG may be accomplished earlier in blood than in urine. In humans, hCG levels may be assessed, for example, at least 10, 11, 12, 13, 14, 15, 16, 17, 18, 19, 20, 21, 22, 23, 24, 25, 26, 27, 28, 29, 30, or more days after fertilization. In humans, hCG levels may be assessed, for example, at least 5, 6, 7, 8, 9, 10, 11, 12, 13, 14, 15, 16, 17, 18, 19, 20, 21, 22, 23, 24, 25, 26, 27, 28, 29, 30, or more days after embryo transfer. Alternatively or additionally, clinical pregnancy may be determined by the presence of fetal cardiac activity. In some embodimetns presence of a fetal cardiac activity is assessed in humans during the ninth week, tenth week, eleventh week, twelth week, thirteenth week, fourtheen week, fifteenth week, sixteenth week, seventeenth week, eighteenth week, nineteenth week, twentieth week, or later post-implantation. In some embodiments, presence of a fetal heartbeat is assessed at 12 weeks and/or during the 12^{th} week post-implantation.

In some embodiments, a positive outcome comprises development of the transferred embryo to a certain fetal stage. Fetal stage may be determined by gestational age, developmental landmarks, or both. For example, development at least through the first trimester or at least through the second trimester of pregnancy a human may be used to indicate a positive outcome. In some embodiments, a positive outcome comprises development of a live fetus at least through 10, 11, 12, 13, 14, 15, 16, 17, 18, 19, 20, 21, 22, 23, 24, 25, 26, 27, 28, 29, 30, 31, 32, 33, 34, 35, 36, 37, or more weeks' gestation. In some embodiments, a positive outcome comprises birth of a live infant; in some such embodiments, a positive outcome comprises birth of a full term live infant. In some embodiments, a positive outcome comprises birth of a full term live infant that is karyotypic ally normal (i.e., has a full and normal set of chromsomes).

### EXAMPLES

### Example 1

### Identification of Markers for In Vitro Fertilization Outcome

Compounds that could be used as markers to predict outcome in in vitro fertilization were identified by analyzing culture media from embryos whose outcomes in IVF procedures were known.

### Materials and Methods

### Sample Collection and Preparation

Embryos that had been fertilized in vitro were grown in culture media. Culture media samples were collected from day 3 embryos and their eventual outcomes after transfer to a female (“positive” or “negative” based on fetal cardiac activity at 12 weeks post-implantation) were recorded. A total of 75 media samples were analyzed in subsequent studies. Each sample contained approximately 30 μL of media and was pooled from culture media of two embryos with similar outcome. Samples were collected from embryos with negative outcomes (25 samples), embryos with positive outcomes (25 samples), and control media with no embryos (12 and 13 samples corresponding to negative and positive outcome embryos respectively).

Samples were received at a laboratory for analysis, inventoried (which included assignment of a unique identifier to each sample that also facilitiated tracking of relationships between samples), and immediately stored at −80° C. until processing.

Sample preparation was carried out using an automated MicroLab STAR® system from the Hamilton Company (Reno, Nev.). Recovery standards were added prior to the first step in the extraction process for QC purposes Immediately before analysis, samples were extracted to remove protein and recover a wide range of chemically diverse compounds. Extraction was performed by shaking for two minutes in the presence of glass beads using a Glen Mills Genogrinder 2000. After extraction, the sample was centrifuged and the supernatant removed using a MicroLab STAR® robotics system. Each extract was split into equal parts for analysis on the gas chromatography (GC) and liquid chromatography (LC) platforms. Samples were placed briefly on a TurboVap® (Zymark) to remove the organic solvent. Each sample was then frozen and dried under vacuum. Samples were then prepared for the appropriate instrument, either LC/MS (liquid chromatography/mass spectrometry) or GC/MS (gas chromatography/mass spectrometry).

### Liquid Chromatography/Mass Spectroscopy (LC/MS)

The LC/MS portion of the platform was based on a Surveyor HPLC and a Thermo-Finnigan LTQ mass spectrometer, which consisted of an electrospray ionization (ESI) source (Fourier transform ion cyclotron resonance (FT-ICR) and linear ion-trap (LIT). Positive and negative ions were monitored within a single analysis by consecutively alternating the ionization polarity of adjacent scans.

Vacuum-dried samples were dissolved in 100 μl of an injection solvent that contained five or more injection standards at fixed concentrations. Internal standards were used to assure both injection and chromatographic consistency. The chromatographic system used a binary solvent system delivered as a gradient. Solvent A was water and solvent B was methanol. Both were high purity grade and both contained 0.1% formic acid as a pH stabilizer. HPLC columns were washed and reconditioned after every injection. All columns were purchased from a single manufacturer's lot. Solvents were similarly purchased in bulk from a single manufacturer's lot in sufficient quantity to complete all related experiments. Raw data files were archived to DVD at regular intervals. Information output from raw data files was extracted as discussed below.

For ions with counts greater than 2 million, an accurate mass measurement could be performed. Accurate mass measurements could be made on the parent ion as well as on fragments. The typical mass error was less than 5 ppm. Ions with less than two million counts require a greater amount of effort to characterize. Fragmentation spectra (MS/MS) were typically generated in data dependent manner, but if necessary, targeted MS/MS could be employed, such as in the case of lower level signals.

### Gas Chromatography/Mass Spectroscopy (GC/MS)

Samples destined for GC/MS analysis were re-dried under vacuum desiccation for a minimum of 24 hours prior to being derivatized under dried nitrogen using bistrimethyl-silyl-triflouroacetamide (BSTFA). The GC column was 5% phenyl and the temperature ramp was from 40° C. to 300° C. in a 16 minute period. Samples were analyzed on a Thermo-Finnigan Trace DSQ fast-scanning single-quadrupole mass spectrometer using electron impact ionization. The instrument was tuned and calibrated for mass resolution and mass accuracy on a daily basis. Information output from raw data files was automatically extracted.

### Bioinformatics

The informatics system comprised four major components: a laboratory Information Management System (LIMS), data extraction and peak-identification software, data processing tools for QC and compound identification, and a collection of information interpretation and visualization tools for use by data analysts. Hardware and software foundations for these informatics components were the LAN backbone and a database server running Oracle 10.2.0.1 Enterprise Edition.

### Data Extraction and Quality Assurance

Data extraction of the raw mass spectroscopy data files yielded information that was loaded into a relational database and manipulated. Once in the database, information was examined and appropriate QC limits were imposed. Peaks were identified using peak integration software, and component parts were stored in a separate and specifically designed complex data structure.

### Compound Identification

Compounds were identified by comparison to library entries of purified standards or recurrent unknown compounds. A combination of chromatographic properties and mass spectra gave an indication of a match to the specific compound or an isobaric compound. Additional compounds could be identified by virtue of their recurrent nature (both chromatographic and mass spectral).

### Curation

A variety of curation procedures were carried out to ensure that a high quality data set was made available for statistical analysis and data interpretation. The QC and curation processes were designed to ensure accurate and consistent identification of true chemical compounds, and to remove those representing system artifacts, mis-assignments, and background noise.

### Normalization

For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”). For studies that did not require more than one day of analysis, no normalization was necessary, other than for purposes of data visualization.

### Statistical Analyses

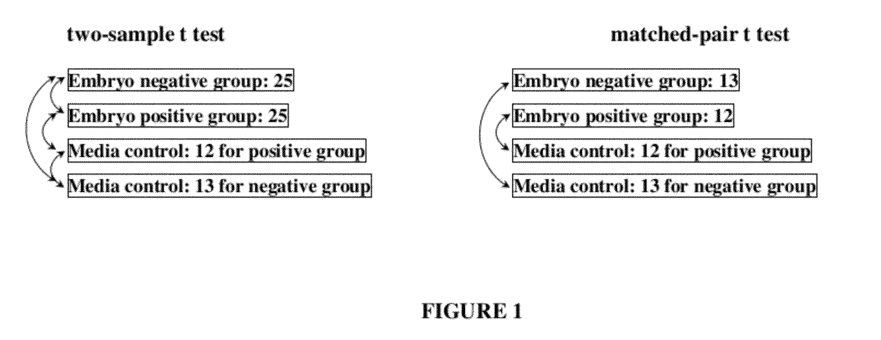

As illustrated in FIG. 1, two sample t-tests and matched pair t-tests were used to analyze the data. Two sample t-test comparisons were performed between the experimental groups.

Twelve samples from each of the positive outcome embryo and negative outcome embryo groups had matching controls. Therefore, matched pair t-tests were used to analyze this subset of samples. For all analyses, missing values (if any) were imputed with the observed minimum for that particular compound. The statistical analyses were performed on natural log-transformed data to account for increases in data variance that occur as the level of response is increased.

Two way ANOVA was also performed in order to generate more statistical power.

For all analyses, both p-values and q-values were calculated for each comparison. p-values indicate the probability of obtaining the values that were observed if the null hypothesis were not true. For example, when comparing samples from negative outcome embryos and samples from positive outcome embryos, the null hypothesis is that amounts of compounds would not differ significantly between the two samples. Lower p-values indicate statistical significance; i.e., increased likelihood that the null hypothesis is not true and should be rejected. q-value indicates the false discovery rate, i.e. a measure of the proportion of false positives that occur when a particular test is considered significant. As with p-values, lower q-values indicate greater significance. A cutoff of p<0.05 was used to identify significantly different values. In some embodiments, a cutoff of p<0.05 may be combined with a cutoff of q<0.2.

### Results

A total of 75 samples from four groups (negative outcome embryos, positive outcome embryos, media only control for negative outcome embryos, and media only control for positive outcome embryos) were analyzed by gas chromatography and liquid chromatography followed by mass spectrometry. Compounds whose amounts differed significantly between groups are presented in Tables 1-4. Table 1 presents results for compounds identified by two sample t-tests as having amounts that differed significantly between groups. Table 2 presents results for compounds identified by matched pair t-tests as having amounts that differed significantly between groups. Table 3 presents results for compounds for which p-values were under 0.05 by two-way ANOVA analysis. Many of the identified compounds were known metabolites. Table 4 summarizes some of the markers identified in Tables 1-3.

Two different comparisons were performed in order to identify markers among the list of compounds. In one comparison, values for positive outcome embryos were compared to values for negative outcome embryos. In another comparison, values for a given group of embryos (e.g., positive or negative outcome) were compared to values for its corresponding control. Detection of a statistically significant difference for one group of embryos but not the other was taken as an indication that the corresponding compound could be used as a marker for embryo outcome.

Compounds with Altered Levels as Determined by Two Sample T-Tests

Two-sample t-test comparisons were performed to test whether mean levels of compounds were different between different experimental groups. Comparisons were performed between
positive outcome embryos vs. media control for positive outcome embryos;
negative outcome embryos vs. media control for negative outcome embryos;
negative outcome embryos vs. positive outcome embryos; and
media control for positive outcome embryos vs. media control for negative outcome embryos.

The fourth comparison listed above, the comparison between different control groups, were performed in order to evaluate variability in data, as media samples were not assumed to be from the same lot of media and/or IVF laboratory.

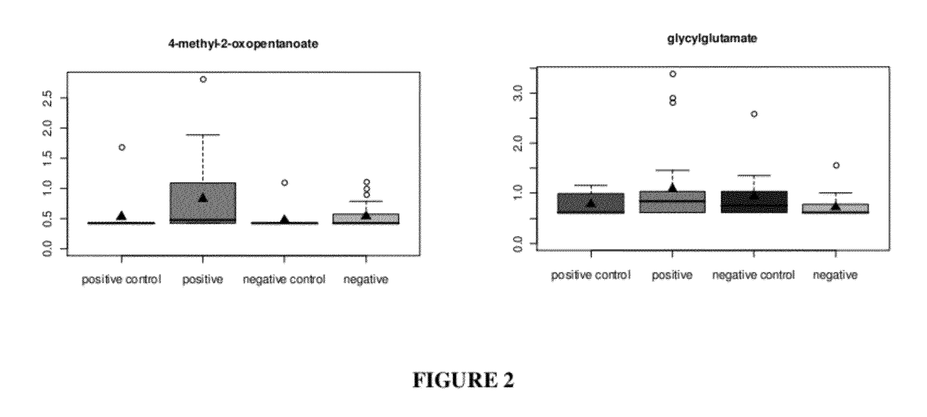

Using the two-sample t-test, 4-methyl-2-oxopentanoate and glycylglutamate were found to be present at significantly higher levels in samples from positive outcome embryos as compared to samples from negative outcome embryos (see Table 1; ratios of values for negative outcome embryos to values for positive outcome embryos are 0.65 and 0.66 for 4-methyl-2-oxopentanoate and glycylglutamate respectively). Significant differences in glycine and 4-methyl-2-oxopentanoate levels were detected between the positive embryo group and its corresponding control group but not between the negative embryo group and its corresponding control group. Glycine levels were slightly but significantly decreased in positive outcome embryos as compared to levels in corresponding controls, whereas 4-methyl-2-oxopentanoate levels were increased in positive outcome embryos as compared to levels in corresponding controls. FIG. 2 presents box plots of values for potential markers identified by the two sample t-test.

Some apparent differences were also observed in the comparison of the two control groups (see Table 1). It is possible that such differences are due to variations in embryo culture procedures and/or media materials at different IVF labs.

TABLE 1
Compounds whose levels were altered per two-sample t-tests

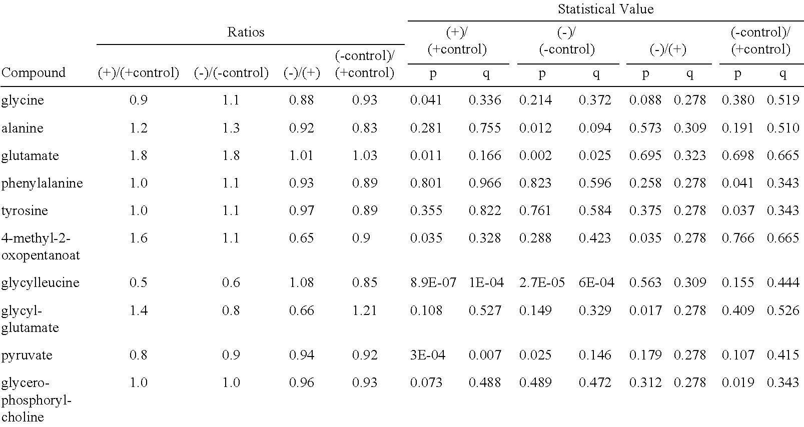

Shaded boxes indicate statistically significant differences

Compounds with Altered Levels Per Two Sample T-Tests

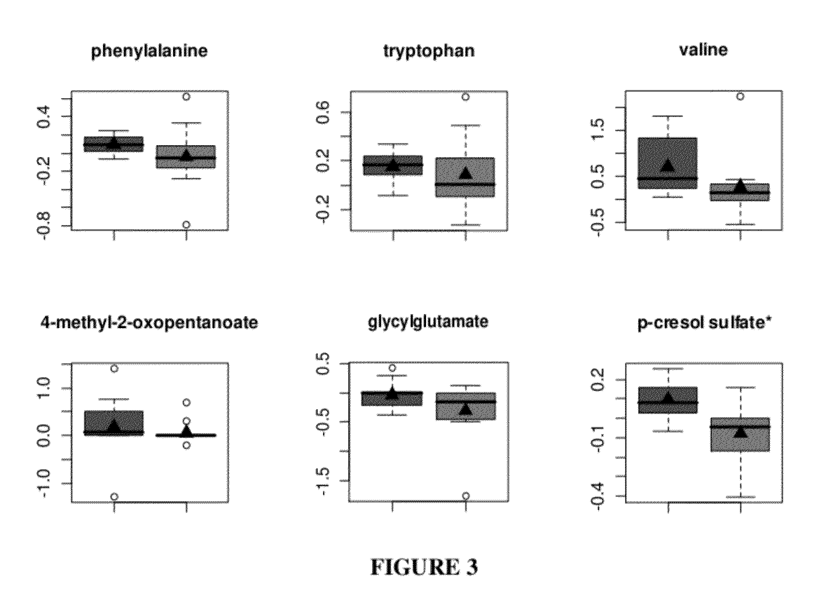

A subset of the samples from embryo groups (12 each from the positive outcome embryo and negative outcome embryo groups) had matching media controls. For these samples, two matched pair t-tests (positive outcome embryo vs. corresponding media control and negative outcome embryos vs. corresponding media control) were performed. Table 2 summarizes results from these analyses. Levels of several compounds (including alanine, phenylalanine, pyruvate, tryptophan, valine, cresol sulfate, and glycylglutamate) were found to be altered significantly in one comparison but not the other. Thus, phenylalanine, tryptophan, valine, cresol sulfate, and glycylglutamate are potential markers for embryo outcome. FIG. 3 presents box plots for each compound, with values for each group of embryos adjusted using their corresponding controls.

The p-value for 4-methyl-2-oxopentanoate in this matched pair t-test was above 0.05. This result is likely due to several outliers among the embryo samples.

TABLE 2
Compounds whose levels were altered per matched pair t-tests

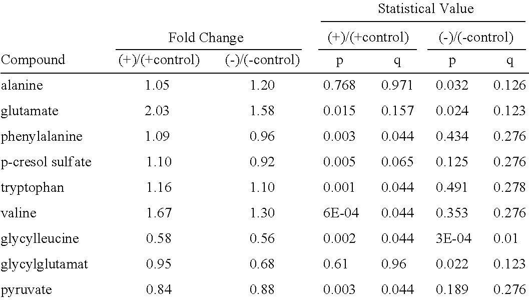

Shaded boxes indicate statistically significant differences

### Two-Way ANOVA to Determine Statistical Significance of Alterations

In an attempt to add greater statistical power to the previous matched pair test analyses, a two way ANOVA was performed to test whether the changes of compound profiles in respect to the controls were different between the positive and negative groups (the ‘outcome x control’ interaction). Several of the compounds identified by the matched pair t-test (FIG. 3) also ranked among the top differentiating compounds in ANOVA analysis (Table 3). Significant differences and a very low rate of false discovery were observed for p-cresol sulfate.

TABLE 3
Significant compounds meeting statistical
cutoff criteria in two-way ANOVA analysis

outcome x control interaction

| Compound | p | q |
|---|---|---|
| p-cresol sulfate | 0.0021 | 0.0755 |
| glycerol 3-phosphate | 0.0066 | 0.1607 |
| glycylglutamate | 0.0266 | 0.3781 |

### Discussion

Based on the above statistical analyses (two sample t-test, matched pair t-test and ANOVA) and the data distribution (separation of the two groups, fold of change, and number of samples detected in each group), the compounds listed in Table 4 are proposed as potential markers to identify embryos that are likely to have a positive outcome.

TABLE 4
Markers for embryo outcome

| | Number of Samples with Detectable Value/ | | | |
|---|---|---|---|---|
| | Total Group Size | | | |
| | | positive | | negative |
| Compound | positive | control | negative | control |
| 4-methyl-2- | 15/25 | 1/12 | 7/25 | 1/13 |
| oxopentanoate | | | | |
| glycylglutamate | 14/25 | 5/12 | 8/25 | 8/13 |
| p-cresol sulfate | 25/25 | 25/25 | 24/25 | 25/25 |
| phenylalanine | 25/25 | 25/25 | 25/25 | 25/25 |
| tryptophan | 25/25 | 25/25 | 24/25 | 25/25 |
| valine | 25/25 | 25/25 | 25/25 | 25/25 |

### EXAMPLE 2

### Prediction of Embryo Outcome Using Compound Profiling of Culture Media

Markers for embryo outcome identified as described in Example 1 can be used to predict the outcome of IVF-implantation for an embryo. For example, embryos can be fertilized in vitro and cultured in preparation for implantation and the culture media collected. Before any embryos are implanted, the culture media can be analyzed for levels of one or more markers (e.g., 4-methyl-2-oxopentanoate, glyculglutamate, phenylalanine, p-cresol sulfate, tryptophan, valine, or a combination thereof). Embryos whose levels of markers match an expected pattern for positive outcome embryos (as determined, for example, in Example 1) may be chosen for implantation into a surrogate, whereas those whose levels of markers match an expected pattern for negative outcome embryos, and/or those whose levels of markers do not match an expected pattern for positive outcome embryos, are not implanted into a surrogate.

Distinguishing embryos with likely positive outcomes from those with likely negative outcomes may allow implantation of a single embryo into a surrogate, thus avoiding the possibility of multiple pregnancies and related complications.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. A method comprising steps of:
providing a sample of culture media in which an embryo has been cultured in vitro;
measuring in the sample of culture media, amount of one or more markers for embryo outcome, wherein the one or more markers comprises a compound selected from the group consisting of 4-methyl-2-oxopentanoate, glycylglutamate, p-cresol sulfate, phenylalanine, tryptophan, valine, and combinations thereof; and
characterizing, on the basis of amount of the one or more markers, whether the embryo is likely to have a positive outcome.

2. The method of claim 1, wherein the embryo developed from a zygote created by in vitro fertilization of an oocyte.

3. The method of claim 1, wherein the embryo developed from a zygote created by intracytoplasmic sperm injection of an oocyte.

4. The method of claim 1, wherein the embryo developed from a zygote created by transfer of a nucleus into an enucleated oocyte.

5. The method of claim 1, wherein the embryo is a mammalian embryo.

6. The method of claim 5, wherein the embryo is a human embryo.

7. The method of claim 5, further comprising transferring the mammalian embryo into the uterine tract of a mammalian female.

8. The method of claim 7, wherein the mammalian embryo is transferred at a developmental stage of at least eight cells.

9. The method of claim 7, wherein the mammalian embryo is transferred at the blastocyst stage.

10. The method of claim 7, wherein the positive outcome comprises implantation into the uterine wall of a mammalian female.

11. The method of claim 7, wherein the positive outcome comprises clinical pregnancy of the mammalian female.

12. The method of claim 7, wherein the positive outcome comprises live birth of an infant that has developed from the transferred embryo.

13. The method of claim 7, wherein the positive outcome comprises development of the transferred embryo to at least the first trimester of pregnancy.

14. The method of claim 7, wherein the positive outcome comprises development of the transferred embryo to at least the first trimester of pregnancy.

15. The method of claim 7, wherein the transferred embryo is transferred with no more than two other embryos.

16. The method of claim 15, wherein the transferred embryo is transferred with no more than one other embryo.

17. The method of claim 16, wherein the transferred embryo is the only embryo transferred.

18. The method of claim 1, wherein the step of measuring amount of one or more markers comprises performing mass spectrometry on the sample of culture media.

19. The method of claim 1, wherein the step of measuring amount of one or more markers comprises performing chromatography on the sample of culture media.

20. The method of claim 19, wherein the step of measuring amount of one or more markers further comprises performing mass spectrometry on the sample of culture media.

21. The method of claim 1, wherein the step of measuring amount of one or more markers comprises comparing amount in the sample of culture media to that in a control.

22. The method of claim 1, further comprising measuring in the sample of culture media, amount of alanine, pyruvate, or both.

23. The method of any one of claims 1, wherein the one or more markers comprises 4-methyl-2-oxopentanoate.

24. The method of any one of claims 1, wherein the one or more markers comprises glycylglutamate

25. The method of any one of claims 1, wherein the one or more markers comprises p-cresol sulfate

26. The method of any one of claims 1, wherein the one or more markers comprises phenylalanine

27. The method of any one of claims 1, wherein the one or more markers comprises tryptophan.

28. The method of any one of claims 1, wherein the one or more markers comprises valine.
